(19) United States
(12) Patent Application Publication
(10) Pub. No.: US 2003/0023211 A1
(43) Pub. Date: Jan. 30, 2003

(54) Neonatal safety net system

(76) Inventor: Marcella M. Eckart, Harvey, ND (US)

Correspondence Address: Michael S. Neustel, 2534 South University Drive, Suite No. 4, Fargo, ND, 58103, US

(21) Appl. No.: 09/912,580

(22) Filed: Jul. 24, 2001

(51) Int. Cl. A61F 13/15

(52) U.S. Cl. 604/358

(57) ABSTRACT

A neonatal safety net system for preventing injury to a baby during childbirth. The neonatal safety net system includes a support member, an extended member extending from the support member, a plurality of end straps attached to the support member, and a plurality of middle straps attached to the support member. The extended member is frictionally positioned between the mother and the delivery table. The support member acts as a safety net when the end straps are connected to an obstetrician. The middle straps are secured around the woman's thighs to further prevent movement of the support member during childbirth.

### BACKGROUND OF THE INVENTION

1. Field of the Invention

The present invention relates generally to neonatal safety devices and more specifically it relates to a neonatal safety net system for preventing injury to a baby during childbirth.

It is estimated that there are over 10,000 childbirths per day in the United States alone. Unfortunately, a number of babies are injured as a result of being accidentally dropped by medical personnel during the childbirth procedure. Hence, there is a need for a system that will reduce the chance of injury to a baby during childbirth.

2. Description of the Prior Art

Examples of patented neonatal safety devices which are illustrative of the prior art include U.S. Pat. No. 5,287,860 to Owens; U.S. Pat. No. 4,963,138 to Braun; U.S. Pat. No. 4,823,418 to Downs; U.S. Pat. No. 4,880,418 to Tramont; U.S. Pat. No. 4,007,741 to Waldrop.

While these devices may be suitable for the particular purpose to which they address, they are not as suitable for preventing injury to a baby during childbirth. Conventional neonatal safety devices are not convenient to utilize nor provide the required safety.

In these respects, the neonatal safety net system according to the present invention substantially departs from the conventional concepts and designs of the prior art, and in so doing provides an apparatus primarily developed for the purpose of preventing injury to a baby during childbirth.

### SUMMARY OF THE INVENTION

In view of the foregoing disadvantages inherent in the known types of neonatal safety devices now present in the prior art, the present invention provides a new neonatal safety net system construction wherein the same can be utilized for preventing injury to a baby during childbirth.

The general purpose of the present invention, which will be described subsequently in greater detail, is to provide a new neonatal safety net system that has many of the advantages of the neonatal safety devices mentioned heretofore and many novel features that result in a new neonatal safety net system which is not anticipated, rendered obvious, suggested, or even implied by any of the prior art neonatal safety devices, either alone or in any combination thereof.

To attain this, the present invention generally comprises a support member, an extended member extending from the support member, a plurality of end straps attached to the support member, and a plurality of middle straps attached to the support member. The extended member is frictionally positioned between the mother and the delivery table. The support member acts as a safety net when the end straps are connected to an obstetrician. The middle straps are secured around the woman's thighs to further prevent movement of the support member during childbirth.

There has thus been outlined, rather broadly, the more important features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a neonatal safety net system that will overcome the shortcomings of the prior art devices.

A second object is to provide a neonatal safety net system for preventing injury to a baby during childbirth.

Another object is to provide a neonatal safety net system that is easily secured to a delivery table.

An additional object is to provide a neonatal safety net system that is easily connected to the attending obstetrician.

A further object is to provide a neonatal safety net system that is comfortably attached to a woman before birthing.

Another object is to provide a neonatal safety net system that provides an added safety mechanism during childbirth to prevent injury to a baby.

A further object is to provide a neonatal safety net system that is able to fit about various sizes of mothers and medical personnel.

Another object is to provide a neonatal safety net system that may be utilized by various individuals such as but not limited to doctors, student interns, nurses, midwifes, missionaries, and other medical personnel.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention.

FIG. 2 is a top view of the present invention.

FIG. 3 is a side view of the present invention.

FIG. 4 is an upper perspective view of the present invention illustrating the connection to the medical professional.

FIG. 5 is a side view of the present invention in use as attached to the medical professional and the mother.

FIG. 6 is a top view of the present invention as attached to the medical professional and the mother.

### DESCRIPTION OF THE PREFERRED EMBODIMENT

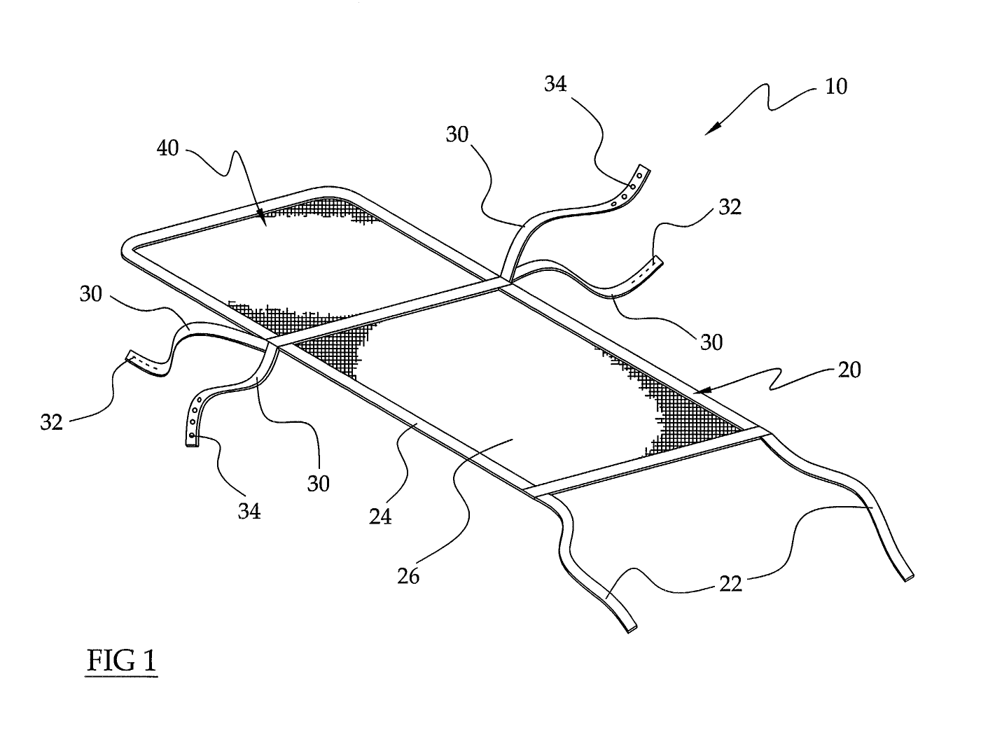

Turning now descriptively to the drawings, in which similar reference characters denote similar elements throughout the several views, FIGS. 1 through 6 illustrate a neonatal safety net system 10, which comprises a support member 20, an extended member 40 extending from the support member 20, a plurality of end straps 22 attached to the support member 20, and a plurality of middle straps 30 attached to the support member 20. The extended member 40 is frictionally positioned between the mother 14 and the delivery table 12. The support member 20 acts as a safety net when the end straps 22 are connected to an obstetrician. The middle straps 30 are secured around the woman's thighs to further prevent movement of the support member 20 during childbirth.

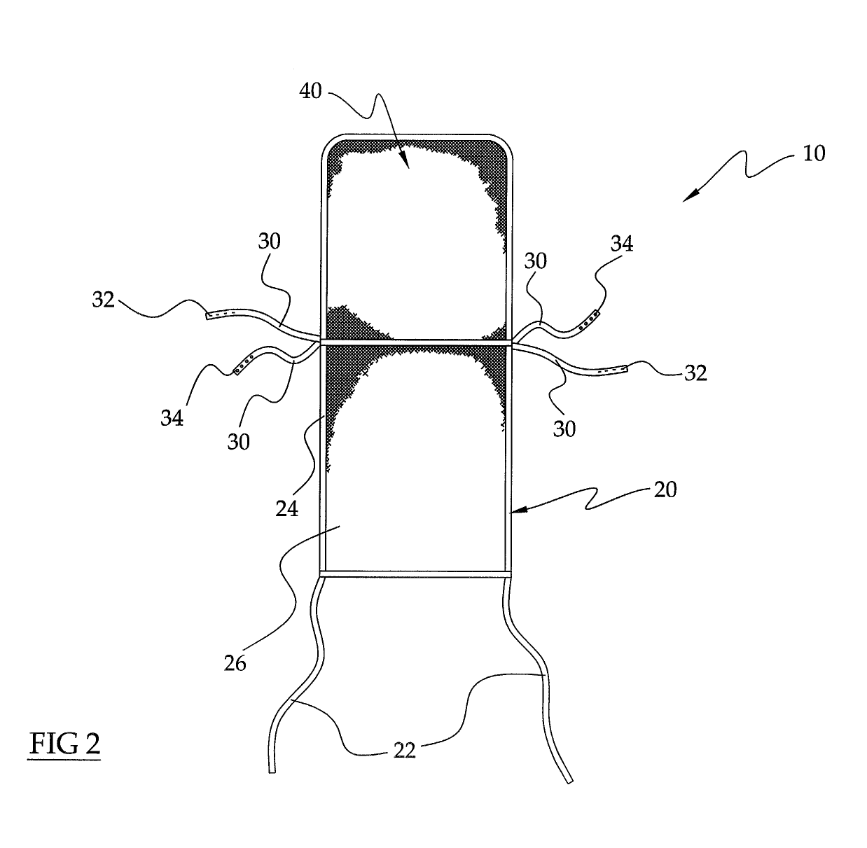

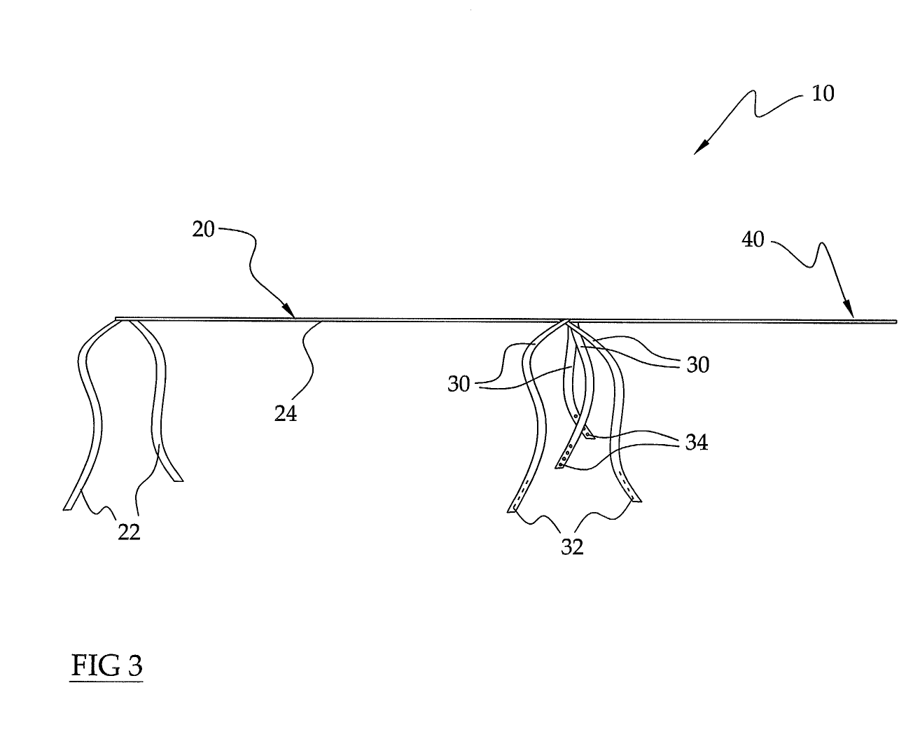

As shown in FIGS. 1, 2 4, 5 and 6 of the drawings, the support member 20 is a generally broad structure. As shown in FIG. 3 of the drawings, the support member 20 preferably has a flat structure. The support member 20 preferably is comprised of a flexible and resilient material which is commonly utilizing within the medical and clothing industries. The support member 20 preferably has a rectangular shape as shown in FIG. 2 of the drawings, however various other shapes may be utilized to construct the support member 20.

As best shown in FIG. 2 of the drawings, the support member 20 is preferably comprised of a sustaining portion 26 and an outer perimeter 24 surrounding the sustaining portion 26 for providing added strength to the sustaining portion 26. It can be appreciated that the outer perimeter 24 is not required to construct and utilize the support member 20 and is shown merely for illustrating an exemplary version of the present invention. The sustaining portion 26 is preferably constructed of a lightweight material such as netting or cloth. It can be appreciated that various other types of materials and structures may be utilized to construct the sustaining portion 26 which are common in the medical and clothing industries.

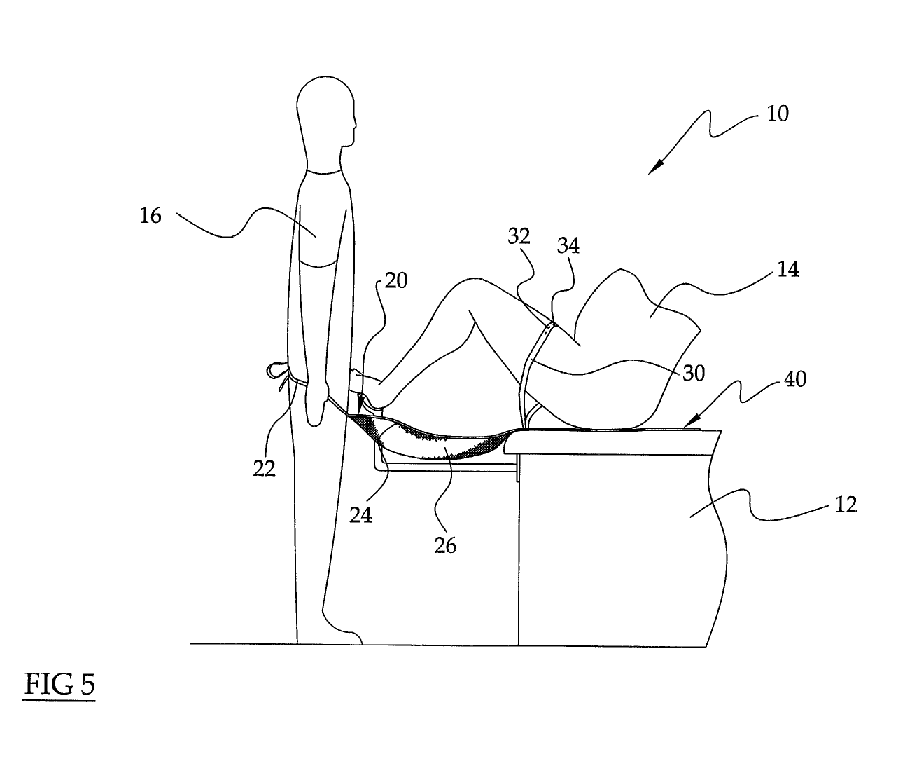

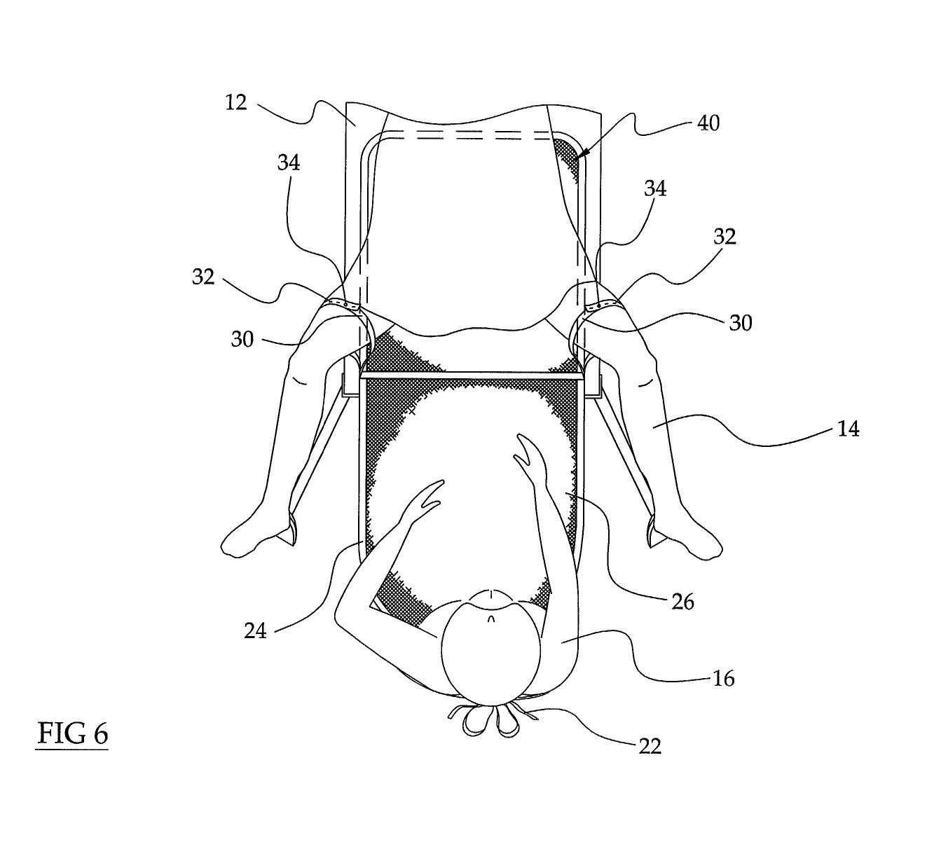

As shown in FIGS. 1 through 6 of the drawings, a plurality of end straps 22 are attached to a distal end of the support member 20. The end straps 22 are utilized for surrounding about the waist of the attending physician or other medical personnel. The end straps 22 may include conventional fasteners for securing the distal portions of the end straps 22 about the waist of the user. The end straps 22 may be constructed so as to be simply tied behind the back of the doctor 16 as shown in FIGS. 5 and 6 of the drawings. Various other attachment means may be utilized for securing the end straps 22 as may be appreciated by one skilled in the art.

As shown in FIGS. 1 through 6 of the drawings, a plurality of middle straps 30 are attached to the support member 20 opposite of the end straps 22. The plurality of middle straps 30 are preferably comprised of a pair of straps on opposing sides of the support member 20 as best illustrated in FIG. 2 of the drawings. The middle straps 30 are constructed for being secured about the legs of the mother 14 during childbirth as shown in FIGS. 5 and 6 of the drawings. The middle straps 30 may include conventional fasteners for securing the distal portions of the middle straps 30 about the legs of the mother 14. The middle straps 30 may be constructed so as to be simply tied about the legs of the mother 14. As best shown in FIG. 1 of the drawings, a plurality of apertures 32 and a plurality of buttons 34 within opposing middle straps 30 provides a suitable system for attaching the middle straps 30 about the legs of the mother 14. However, various other attachment means may be utilized for securing the middle straps 30 as may be appreciated by one skilled in the art.

As shown in FIGS. 1 through 6 of the drawings, an extended member 40 is attached to the support member 20 adjacent the middle straps 30 and opposite of the end straps 22. The extended member 40 is constructed of a material and structure similar to the support member 20. The extended member 40 is designed for being positioned between the lower portion of the mother 14 and the delivery table 12 as best illustrated in FIGS. 5 and 6 of the drawings. The extended member 40 may have a width equal to, less than or greater than the support member 20 as can be appreciated.

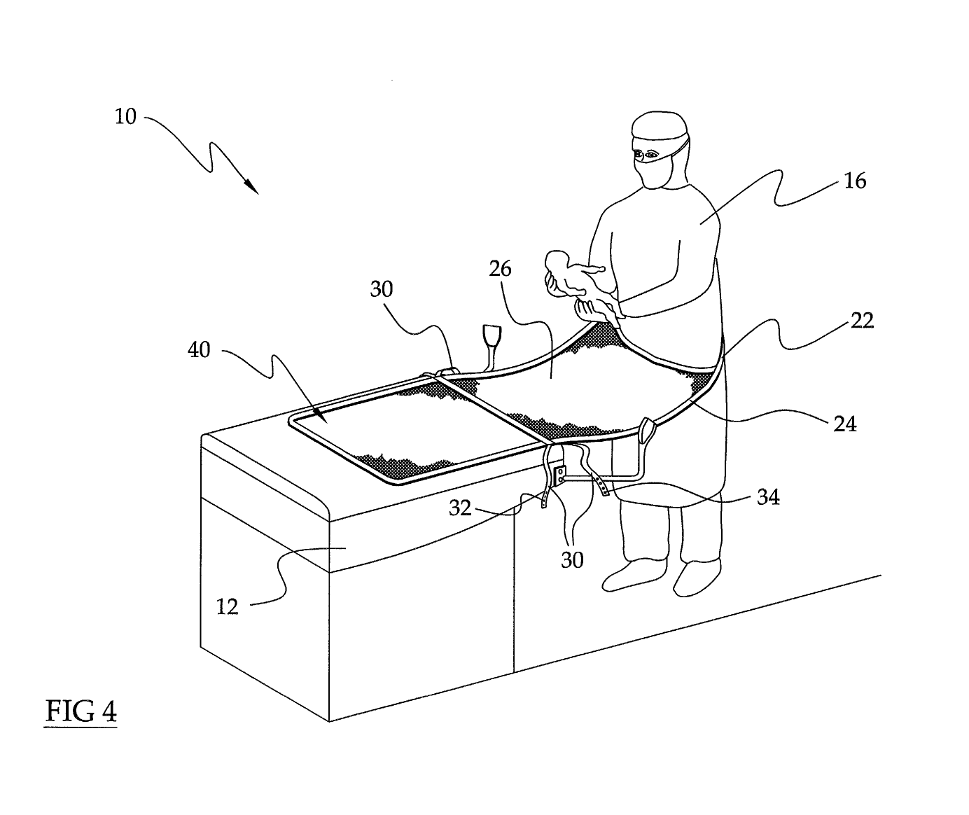

In use, the user positions the extended member 40 upon the upper surface of the delivery table 12 as shown in FIG. 4 of the drawings. The mother 14 is then positioned upon the delivery table 12 and upon the extended member 40 as shown in FIGS. 5 and 6 of the drawings. The weight of the mother 14 frictionally retains the extended member 40 in the desired position. The middle straps 30 are then secured about the legs of the mother 14 as further shown in FIGS. 5 and 6 of the drawings. The support member 20 is then placed to the side of the patient during the early stages of childbirth. At the time the mother 14 is able to give birth to the baby, the attending doctor 16 or other individual assisting in the birth then secures the end straps 22 about their waste as shown in FIGS. 4 through 6 of the drawings. The support member 20 is then “stretched” between the doctor 16 and the mother 14 in the lower pelvis area between the legs of the mother 14 thereby “catching” an accidentally dropped baby during childbirth. When the baby has been delivered, the end straps 22 and the middle straps 30 are simply removed and the above process reversed.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed to be within the expertise of those skilled in the art, and all equivalent structural variations and relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A neonatal safety net system for extending between a patient and a delivery person for preventing accidentally dropping a baby during childbirth, comprising:
a support member having a broad structure capable of receiving a baby;
a plurality of first straps attached to a distal end of said support member, wherein said plurality of first straps are formed for securing about the waist of said delivery person;
a plurality of second straps attached to said support member opposite of said first straps, wherein said plurality of second straps are formed for securing about the legs of said patient; and
an extended member attached to a distal end of said support member opposite of said plurality of first straps, wherein said extended member is formed for being positioned between a lower portion of said patient and a delivery table.

2. The neonatal safety net system of claim 1, wherein said support member is comprised of a flexible and resilient material.

3. The neonatal safety net system of claim 2, wherein said support member is comprised of a netting material.

4. The neonatal safety net system of claim 3, wherein said support member includes an outer perimeter surrounding a sustaining portion.

5. The neonatal safety net system of claim 1, wherein said support member is comprised of a netting material.

6. The neonatal safety net system of claim 1, wherein said extended member has a width lesser than said support member.

7. The neonatal safety net system of claim 1, wherein said plurality of first straps are comprised of a pair of first straps.

8. The neonatal safety net system of claim 1, wherein said plurality of second straps are comprised of two pairs of second straps.

9. The neonatal safety net system of claim 8, wherein each of said two pairs of second straps include a securing means for securing said two pairs of second straps about the legs of said patient.

10. The neonatal safety net system of claim 9, wherein said securing means is comprised of a plurality of apertures within one of said second straps and a plurality of buttons within another of said second straps.

11. A neonatal safety net system for extending between a patient and a delivery person for preventing accidentally dropping a baby during childbirth, comprising:
a support member having a broad rectangular structure capable of receiving a baby, wherein said support member has a width less than a width of a delivery table;
a plurality of first straps attached to a distal end of said support member, wherein said plurality of first straps are formed for securing about the waist of said delivery person;
a plurality of second straps attached to said support member opposite of said first straps, wherein said plurality of second straps are formed for securing about the legs of said patient; and
an extended member attached to a distal end of said support member opposite of said plurality of first straps, wherein said extended member is formed for being positioned between a lower portion of said patient and said delivery table.

12. The neonatal safety net system of claim 11, wherein said support member is comprised of a flexible and resilient material.

13. The neonatal safety net system of claim 12, wherein said support member is comprised of a netting material.

14. The neonatal safety net system of claim 13, wherein said support member includes an outer perimeter surrounding a sustaining portion.

15. The neonatal safety net system of claim 11, wherein said support member is comprised of a netting material.

16. The neonatal safety net system of claim 11, wherein said extended member has a width lesser than said support member.

17. The neonatal safety net system of claim 11, wherein said plurality of first straps are comprised of a pair of first straps.

18. The neonatal safety net system of claim 11, wherein said plurality of second straps are comprised of two pairs of second straps.

19. The neonatal safety net system of claim 18, wherein each of said two pairs of second straps include a securing means for securing said two pairs of second straps about the legs of said patient.

20. The neonatal safety net system of claim 19, wherein said securing means is comprised of a plurality of apertures within one of said second straps and a plurality of buttons within another of said second straps.
